(19) United States
(12) Patent Application Publication
(10) Pub. No.: US 2006/0241696 A1
(43) Pub. Date: Oct. 26, 2006

(54) Method of limiting hair loss and promoting hair growth

(76) Inventor: Stjepan Krco, Bronx, NY (US)

Correspondence Address: THE FIRM OF KARL F ROSS, 5676 RIVERDALE AVENUE, PO BOX 900, RIVERDALE (BRONX), NY, 10471-0900, US

(21) Appl. No.: 11/118,847

(22) Filed: Apr. 26, 2005

(51) Int. Cl. A61N 1/39

(52) U.S. Cl. 607/1

(57) ABSTRACT

A method of treating baldness or promoting hair grown or limiting hair loss in which a scalp region is subjected to removal of at least the epidermal layer. The epidermal layer can be removed by a chemical skin removal, a laser skin removal procedure, a radiofrequency ablation, abrasion, jet peeling, scalpel, by the use of a dermatome, by a burning operation.

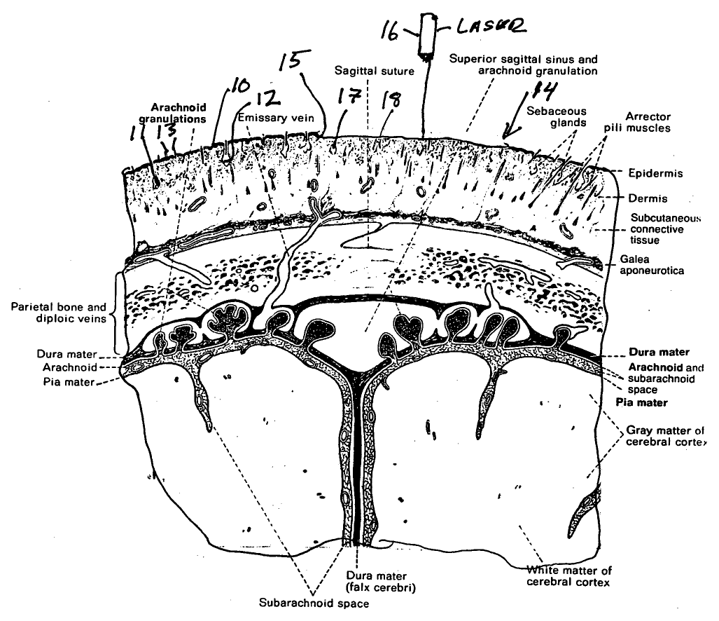

### FIELD OF THE INVENTION

My present invention relates to a method of limiting hair loss and/or promoting hair growth. More particularly, the invention relates to a treatment of the scalp to prevent hair loss or promote follicular growth.

### BACKGROUND OF THE INVENTION

While numerous techniques have been alleged to be effective over many generations for preventing hair loss and especially male-pattern baldness, alopecia, follicle damage from injury, infection or inflammation, or diffuse hair loss especially in women or to promote hair growth in regions of the scalp which have been rendered devoid of hair as a result of male pattern baldness or the other indicia mentioned, few of the techniques described anecdotally as capable of promoting hair grown or limiting male pattern baldness have actually been found to be effective in practice.

Androgenetic alopecia or androgenic alopecia or seborrheic alopecia or common-alopecia or male/female pattern hair loss (PHL) is a specific type of alopecia that is characterized by progressive miniaturization of scalp hair follicles.

Two, FDA approved, drugs (minoxidil solution and finasteride orally) have been used to stabilize hair loss and promote hair regrowth. Microtransplant of follicular units and of minigrafts and micrografts is currently most advanced surgical option.

The prevalence of PHL in the United States is more common than previously reported and is still rising. (Rhodes T. Girman C J, Savin R C, Kaufman K D, Guo S. Lilly F R, Siervogel R M, Chumlea W C. Prevalence of male pattern hair loss in 18-49 year old men. Dermatol Surg 1998 December; 24 (12): 1330-2).

The prevalence of PHL among Caucasians has been estimated to be 30% of men by the age of 30, 50% of men by the age of 50, and 50% of women by the age of 60. This estimate is for current prevalence in developed part of the world. There is no reliable data of prevalence of PHL in underdeveloped or rural part of the World although are some indications that prevalence of PHL in this part is significantly lower. There are many indications that this prevalence was significantly lower in the past in the developed World than presently.

I have found a great influence of moderate temperature on the pathogenesis of PHL. I believe PHL is to be a consequence of repeated application of high shower water temperature over long periods of time. The high shower water temperature may cause thermal injury to scalp skin with many morphological, histological, and physiological effects.

Pattern hair loss is often classified as reversible considering that hair follicles are present and cycling even in a bald scalp (Sinclair R D, Dawber R P. Androgenetic alopecia in men and women. Clin Dermatol. 2001 March-April; 19(2):167-78).

Miniaturized hair follicles of pattern alopecia can quickly regenerate once removed from the human scalp and can grow as well as or better than terminal follicles from the same individual (Krajcik R A, Vogelman J H, Malloy V L, Orentreich N. Transplants from balding and hairy androgenetic alopecia scalp regrow hair comparably well on immunodeficient mice. J Am Acad Dermatol. 2003 May; 48(5): 752-9)

On vertical and horizontal scalp biopsies a reduction is visible in the number of terminal anagen hairs. These hairs are replaced by secondary pseudo vallus hairs with residual angiofibrotic follicular streamers or stellae. The density of follicles does not appear to be noticeably diminished. Occasional follicles are unequivocally destroyed by the intense inflammatory infiltrate (in late PHL). (KLIGMAN A M. The comparative histopathology of male pattern baldness and senescent baldness. Clin Dermatol 1988; 6:18-18).

Many pseudo vallus follicles can be identified in the dermis, indicating that follicles are miniaturized rather than destroyed. Pseudo vallus hairs are in a presence of arrector pili muscle and angiofibrotic streamers as a difference to regular vellus hairs. (Sinclair R D, Dawber R P. Androgenetic alopecia in men and women. Clin Dermatol. 2001 March-April; 19(2): 167-78).

Count of small or vellus follicles must be done in transverse section within a 1 mm of the undersurface of the epidermis to obtain a rate count. The normal density of follicular units in the adult human scalp as counted in the middermal zone is about 1/mm². The hair bulb may be encountered at almost any level from the high-dermis to the subcutis. In the subcutis, such bulbs usually belong to large terminal hairs. In the high dermis, they usually are of small diameter and belong to vellus hairs. (Headington Jt. Transverse microscopic anatomy of the human scalp. A basis for a morphometric approach to disorders of the hair follicle is described in Arch Dermatol 1984 April; 120(4): 449-56.

Cutaneous wound healing is a complex process encompassing a number of overlapping events that include leukocyte recruitment, matrix deposition, epitheliaziation, and ultimately resolution of inflammation with formation of mature scar. Stem cells have high proliferative potential at a time of tissue expansion like wound healing. Results here demonstrate that epithelial stem cells were located in the bulge and not in the lower follicle (although some isolated positive cells were found there) (Lyle S, Christofidou-Solomidou M, Liu Y, Elder D E, Albelda S, Costarelis G. The c8/144B monoclonal antibody recognizes cytokeratin 15 and defines the location of human hair follicle stem cells. J. Cell Sci 1998 November; 111 (Pt 21): 3179-3188).

Reynolds A J, and Jahoda C A. Inductive properties of hair follicle cells. Ann NY Acad Sci. 1991 Dec. 26; 642:226-41 have shown that in the absence of the dermal papilla, cells from the germinative epithelium can induce cells from the dermal sheath to become dermal papilla cells and re-form new follicle.

Oliver demonstrated that cells of the human follicle have regenerative and inductive abilities. These abilities are required because their normal cycling involves repeated cell and tissue interaction and remodeling. (Jahoda C A, Oliver R F, Reynolds A J, Forrester J C, Horne K A. Human hair follicle regeneration following amputation and grafting into the nude mouse. J. Invest Dermatol. 1996 December; 107(6): 804-7.

Several groups have investigated whether papillae alter their expression in follicles from bald scalp skin. Most studies have concluded that dermal papilla is essentially normal (Allegra, Lattanand, Johnson W C. Male pattern alopecia a histopathologic and histochemical study. J Cutan Pathol 1975; 2(2):58-70).

Human follicles have been shown restored by lower follicle or connective tissue sheath cells (Jahoda C A, Oliver R F, Reynolds A J, Forrester J C, Horne K A. Human hair follicle regeneration following amputation and grafting into the nude mouse. J Invest Dermatol. 1996 December; 107(6):804-7).

The thickness of the dermis, hypodermis and galea capitis of the scalp skin varied with gender and age of subject. The thickness of the epidermis, dermis and hypodermis in a balding subject was significantly decreased by comparison with those in a normal subject. (Hori H, Moretti G, Rebora A, Crovato F. The thickness of human scalp: normal and bald. J Invest Dermatol. 1972 June; 58(6):396-9).

Dermal papilla-dermal sheath cell transfer can be bi-directional: as the sheath cells become papilla cells so papilla cells must regenerate dermal sheath cells (Reynolds A J, Jahoda C A. Cultured dermal papilla sells induce follicle formation and hair growth by transdifferentitation of an adult epidermis. Development. 1992 June; 115(2):587-93).

Horne K A, Jahoda C A B. Restoration of hair growth by surgical implantation of follicular dermal sheath. Development 1992 November; 116(3):563-571. Have shown that implanted lower mesenchyme sheath formed completely new dermal papilla and epidermal matrix complex. The sheath/germinative and papilla/germinative cell implants repeatedly induced larger and more frequent vibrissa-type follicles and fibers. Pilosebaceous units induced from DS/GE cell implants. When influenced by GE cells interaction, Ds cells can specialize and adopt an ‘inductive status’ while ‘aged’ DP cells can be revitalized so that their inductive capacity is restored. (Reynolds A J, Jahoda C A B. Hair matrix germinative epidermal cells confer follicle-inducing capabilities on dermal sheath and high passage papilla cells. Development 1996; 122:3085-3094).

By combining follicle dermal sheath and outer root sheath cells in organotypic chambers, a skin equivalent has been created with characteristic dermal and epidermal architecture and a normal basement membrane—the first skin to be produced entirely from hair follicle cells (Gharzi A, Reynolds A J, Jahoda C A B. Plasticity of hair follicle dermal cells in wound healing and induction. Exp Dermatol 2003:12:126-136).

Tufted hair folliculitis is characterized by the emergence of multiple hairs from widely dilated folicular orifices on bald or balding scalp (Jappe U, Schroder X, Zillikens D, Petzoldt D. Tufted hair folliculitis associated with pemphigus vulgaris. J Eur Acad Dermatol Venerol 2003 March;17(2):223-6).

It has long been recognized that new epidermis can be derived from skin appendages after skin injury.

Existence of multiple stem cells in cutaneous epithelium. Loss of stem cell in one proliferative unit is replaced by stem cells in the adjacent unit (Ghazizadeh S, Taichman L B. Multiple classes of stem cells in cutaneous epithelium; a lineage analysis of adult mouse skin. EMBO J. 2001 Mar. 15; 20(6);1215-1222).

Thus there are suggestions in the art as to the application of growth promoting chemicals topically to the scalp, stimulation techniques, grafting or combinations of chemical and mechanical treatments.

What has been found to be effective with respect to specific and selective hair growth is implantation by, for example, making incisions in the scalp and inserting hair follicles into these incisions. The method, while effective is very time consuming and expensive, is only limitedly effective for many patients, can be prohibitively painful and can result in infection or other scalp damage.

### OBJECTS OF THE INVENTION

It is the principal object of the present invention to provide an improved method of reducing hair loss and promoting hair growth.

Another object of the invention is to provide an improved dermatological treatment which can treat or prevent hair loss and/or can restore hair to a scalp region denuded of air without the drawbacks of earlier methods.

It is also an object of the invention to provide a dermatological treatment for baldness, alopecia, seborrhea and acne which may be more effective than earlier treatments.

### SUMMARY OF THE INVENTION

These objects and others which will become apparent hereinafter are attained, in accordance with the invention in a method of treating baldness which comprises the step of removing at least an epidermal layer of a scalp of a patient without damage to hair follicles in a treated region and to a depth sufficient to promote hair growth in that region.

The invention is based upon my discovery that, apparently, male pattern baldness in particular but baldness in generally may be a consequence of some microscopic and not readily observable or observed damage to the epidermis and dermis of the scalp which inhibits the growth of hair follicles or causes hair loss. For example, I have observed that excessive exposure of the scalp to hot water appears to cause microscopic damage to the epidermis of the scalp and is most pronounced in subjects utilizing excessive temperatures in shower water or washing ones head excessively with hot water. Indeed, an investigation which has been made comparing subjects using hotter water or greater exposures of the scalp to hotter water, it has been found that subjects whose scalps have been thus exposed tend to suffer greater hair loss, normally associated with male pattern baldness than subjects whose scalps have been less exposed to hot water. I have also observed that surprising hair growth phenomena can arise where there is a removal of at least an epidermal layer of the scalp with respect to hair growth in such regions.

According to the invention a layer of the scalp is removed of a thickness at least equal to the thickness of the epidermis and, preferably, slightly, more so than the removal extends to a limited extent into the dermis of the scalp of the patient. The layer removed, can have a thickness of at least 1 mm, although smaller thicknesses have been found to be effective in some cases. The removal of this portion of the scalp of the patient, i.e. At least the epidermal layer, can be effected by a chemical skin removal procedure, a laser skin removal procedure, a radio-frequency ablation, by the use of a dermatome, fluid-jet peeling, by a burning operation, by scalpel or by abrasion.

The invention also comprises a method of limiting hair loss and restoring hair to a scalp region of a patient which comprises restricting contact of the scalp region of the patient with hot water having a temperature in excess of 95° C. over a prolonged period over a minimum of three months and sufficient at least to initiate regrowth of hair in the treated region. The maximum temperature to which this regions should be exposed over this period is preferably 88° F.

The latter method may also include the step of removing at least an epidermal layer of the scalp of the patient without damage to hair follicles to a depth sufficient to promote hair growth in the region prior to, subsequent to or during the treatment.

The invention is also a dermatological treatment for baldness, alopecia, seborrhea or acne. In particular I have found that the removal of at least the epidermal layer as described is effective for the treatment of seborrhea and acne as well. Here again the thickness of the layer removed is at least equal to the thickness of the epidermis, is preferably at least 1 mm and can extend into the dermis of the patient. Any of the above-mentioned techniques for skin removal may be used here as well.

While the method of the invention is applicable to male and female adults, there is reason to believe that it is applicble to children as well.

### BRIEF DESCRIPTION OF THE DRAWING

The above and other objects, features, and advantages will become more readily apparent from the following description, reference being made to the accompanying drawing, the sole FIGURE of which is a cross section through the scalp of a patient illustrating the principles of the invention.

### SPECIFIC DESCRIPTION

In the drawing I have shown a cross sectional view through the scalp of the patient in which the epidermis is visible at 10 and below the epidermis the dermis can be seen at 11. Hair follicles 12 which extend from the dermis through the epidermis may have remaining hair at 13. In a region 14 in which hair has been lost, the epidermis and a small portion of the dermis are removed at 15 by for example the action of a laser 14 or any of the other means described. From the follicles 17 in the dermis below the surface 18 resulting from the removal of the epidermal layer, new hair can grow. The treated regions ere permitted to heal without furthhter irritation.

Preferably the patient thus treated also follows a restricted routine of bathing without showering with water at a temperature of 88° F. or higher.

I claim:

1. A method of treating baldness comprising the step of removing at least an epidermal layer of a scalp of a patient without damage to hair follicles to a depth sufficient to promote hair growth in a treated region.

2. The method defined in claim 1 wherein a layer having a thickness at least equal to the thickness of the epidermis of the scalp of the patient is removed.

3. The method defined in claim 2 wherein the layer removed has a thickness of at least 1 mm.

4. The method defined in claim 3 wherein said layer is of sufficient thickness to include part of the dermis of the scalp if the patient.

5. The method defined in claim 4 wherein said layer is removed by at least one of a chemical skin removal, a laser skin removal, a radiofrequency ablation, abrasion, jet peeling, scalpel, a dermatome and a burning operation.

6. A method of limiting hair loss and restoring hair to a scalp region of a patient which comprises restricting contact of the scalp region of the patient with hot water having a temperature in excess of 95° F. over a prolonged period of a minimum of three months and sufficient at least to initiate regrowth of hair in said region.

7. The method defined in claim 6, further comprising the step of removing at least an epidermal layer of a scalp of a patient without damage to hair follicles to a depth sufficient to promote hair growth in the treated region prior to, subsequent to or during said period.

8. A dermatological treatment for baldness, alopecia, seborrhea and acne comprising removing at least an epidermal layer of a scalp of a patient.

9. The method defined in claim 8 wherein a layer having a thickness at least equal to the thickness of the epidermis of the scalp of the patient is removed.

10. The method defined in claim 9 wherein the layer removed has a thickness of at least 1 mm.

11. The method defined in claim 10 wherein said layer is of sufficient thickness to include part of the dermis of the scalp if the patient.

12. The method defined in claim 11 wherein said layer is removed by at least one of a chemical skin removal, a laser skin removal, a radiofrequency ablation, abrasion, jet peeling, scalpel, a dermatome and a burning operation.
